(19) United States
(12) Patent Application Publication
(10) Pub. No.: US 2006/0184194 A1
(43) Pub. Date: Aug. 17, 2006

(54) Embolic protection device

(75) Inventors: Dharmendra Pal, Wilmington, MA (US); Thomas A. Osborne, Bloomington, IN (US); Ram H. Paul JR., Bloomington, IN (US)

Correspondence Address: BRINKS HOFER GILSON & LIONE/CHICAGO/COOK, PO BOX 10395, CHICAGO, IL, 60610, US

(73) Assignee: Cook Incorporated, Bloomington, IN (US)

(21) Appl. No.: 11/353,380

(22) Filed: Feb. 14, 2006

Related U.S. Application Data
(60) Provisional application No. 60/652,956, filed on Feb. 15, 2005.

(51) Int. Cl. A61M 29/00

(52) U.S. Cl. 606/200

(57) ABSTRACT

An embolic protection device generally includes an elongated hub and at least one projection. The projection defines a tortuous pathway between the hub and the vessel wall to prevent emboli from following the pathway and passing beyond the device. The tortuous pathway is structured to permit the bodily fluid to follow the pathway and pass beyond the device.

### CROSS-REFERENCE TO RELATED APPLICATION

This application claims the benefit of U.S. Provisional Application Ser. No. 60/652,956, filed on Feb. 15, 2005, entitled “EMBOLIC PROTECTION DEVICE,” the entire contents of which are incorporated herein by reference.

### FIELD OF THE INVENTION

The present invention relates to medical devices. More particularly, the present invention relates to emboli capturing devices and methods for capturing emboli within a body lumen.

### BACKGROUND OF THE INVENTION

Embolic protection devices are percutaneously placed in a body lumen to prevent emboli from traveling and creating an undesirable embolism, e.g., pulmonary embolism. For example, vena cava filters are being used for trapping emboli in the vena cava to prevent pulmonary embolism. There are many potential sources of undesirable emboli.

For Example, treatments for a stenotic lesion provide a potential in releasing blood clots and other thrombi plaque in the vasculature of the patient. One common procedure is the treatment of a carotid artery stenosis. Generally, carotid artery stenosis is the narrowing of the carotid arteries, the main arteries in the neck that supply blood to the brain. Carotid artery stenosis (also called carotid artery disease) is a relatively high risk factor for ischemic stroke. The narrowing is usually caused by plaque build-up in the carotid artery.

Carotid angioplasty is a more recently developed treatment for carotid artery stenosis. This treatment uses balloons and/or stents to open a narrowed artery. Carotid angioplasty is a procedure that can be performed via a standard percutaneous transfemoral approach with the patient anesthetized using light intravenous sedation. At the stenosis area, an angioplasty balloon is delivered to predilate the stenosis in preparation for stent placement. The balloon is then removed and exchanged via catheter for a stent delivery device. Once in position, a stent is deployed across the stenotic area. If needed, an additional balloon can be placed inside the deployed stent for post-dilation to make sure the struts of the stent are pressed firmly against the inner surface of the vessel wall. During the stenosis procedure however, there is a risk of such blood clots and thrombi being undesirably released into the blood flow within the vasculature.

Accordingly, there is a need to provide a device and method for capturing emboli within a body lumen, including providing distal protection during a procedure that has the potential for emboli.

### BRIEF SUMMARY OF THE INVENTION

One embodiment of the present invention provides an embolic protection device designed to be positioned inside a body vessel having bodily fluid to capture emboli within the vessel. The embolic protection device generally includes an elongated hub and at least one projection. The projection extends radially from the hub as well as longitudinally along the hub. The at least one projection defines a tortuous pathway between the hub and the vessel wall. The tortuous pathway is structured to prevent emboli from following the pathway and passing beyond the device. The tortuous pathway is structured to permit the bodily fluid to follow the pathway and pass beyond the device.

According to more detailed aspects, the at least one projection preferably comprises a plurality of bristles attached to the hub. The bristles are attached to the hub along a spiral path to give the tortuous pathway a spiral shape, or may take other torturous shapes to form pathways suitable for capturing emboli. For example, when the bodily fluid is blood, the tortuous pathway is structured to permit blood cells to follow the pathway, while emboli will be incapable of following the pathway and will be captured by the bristles.

The plurality of bristles or other projection is operable between a loaded position and a deployed position. The bristles are biased towards the deployed position and move radially outwardly when moving from the loaded position to the deployed position. Preferably, the device is used in conjunction with a catheter, and the hub and projection bristles are fitted inside the catheter in the loaded position. The catheter is then moved relative to the hub to expose the projection/bristles and permit the same to expand into the deployed position. A reverse procedure may be employed for re-loading the device. In another embodiment, an actuating member is be employed to operate the projection between its loaded and deployed conditions, and the projection may be un-biased or biased to its loaded position.

Most preferably, the catheter is a balloon catheter for treating a stenotic lesion, and the hub and bristles provide distal protection for the balloon catheter. The hub may be formed as a wire guide or simply a metal or plastic tether. The hub may also include a lumen sized to receive a wire guide for use in conjunction therewith.

### BRIEF DESCRIPTION OF THE DRAWINGS

The accompanying drawings incorporated in and forming a part of the specification illustrate several aspects of the present invention, and together with the description serve to explain the principles of the invention. In the drawings:

FIG. 1 is a partial cross-sectional view, depicting an embodiment of an embolic protection device constructed in accordance with the teachings of the present invention;

FIG. 2 is a partial cross-sectional view of the embolic protection device depicted in FIG. 1, shown in its loaded condition;

FIG. 3 is a partial cross-sectional view of another embodiment of an embolic protection device constructed in accordance with the teachings of the present invention;

FIG. 4 is an end view of a hub forming a portion of the embolic protection device depicted in FIGS. 1-7;

FIG. 5 is a partial cross-sectional view of another embodiment of an embolic protection device constructed in accordance with the teachings of the present invention;

FIG. 6 is a partial cross-sectional view of another embodiment of an embolic protection device constructed in accordance with the teachings of the present invention; and

FIG. 7 is a partial cross-sectional view of the embolic protection device depicted in FIG. 6, shown transitioning between its loaded and deployed conditions.

### DETAILED DESCRIPTION OF THE INVENTION

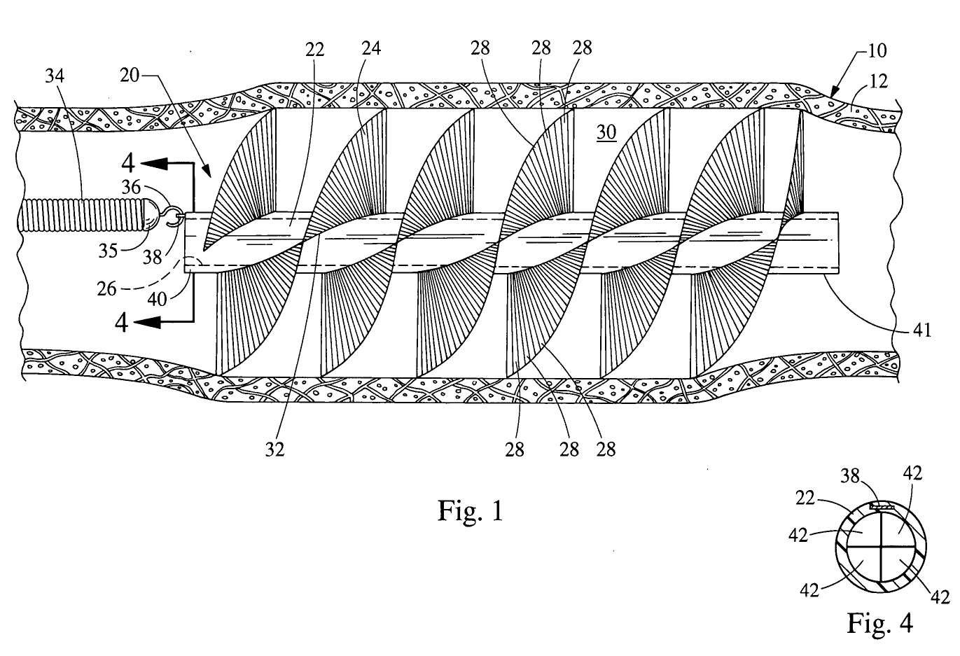

Turning now to the figures, FIG. 1 depicts a cross-sectional view of the embolic protection device 20 constructed in accordance with the teachings of the present invention. The device 20 is designed to be positioned inside a body lumen 10 which has a bodily fluid passing therethrough, such as a blood vessel. The bodily vessel 10 is defined by a vessel wall 12. The embolic protection device 20 generally comprises an elongated hub 22 and at least one projection 24 extending radially from the hub. As shown in the figures, the at least one projection 24 may comprise a plurality of bristles 28 which are attached to the outer periphery of the hub 22. The bristles are preferably constructed of a biocompatible plastic such as polyamide, polytetrafluroethylene (PPFE), polyethylene (LDPE, HDPE) or polyvinylchloride (PVC), although may be constructed of other known materials such as metals (e.g. nitinol (Ni-Ti superelastic alloy) or other alloys) other plastics, elastomers, or composites. The hub 22 is also preferably constructed of similar material, but may be of a material different from the bristles 28.

In FIG. 1 the embolic protection device 20 is shown in its deployed condition where the plurality of bristles 28 extend radially outwardly and engage the wall 12 of the body vessel 10. In this manner, the projection 24 defined by the plurality of bristles 28 forms a tortuous pathway 30 between the hub 22 and the vessel wall 12. As shown, the tortuous pathway 30 has been depicted as having a spiral configuration, although other tortuous pathways can readily be employed as will be envisioned by those of skill in the art. The plurality of bristles 28 are attached to the hub 22 along a spiral path 32 which traverses the outer periphery and extends longitudinally down the hub 22 to form the spiral tortuous path 30.

The embolic protection device 20 is thus capable of collecting and trapping emboli by virtue of the flow characteristics of emboli and bodily fluid flowing through the body vessel 10. That is, the larger the emboli the more laminar and centralized its flow through the body vessel 10. To the contrary, the bodily fluid is capable of following a non-laminar flow such as turbulent flow or a spiral flow through the embolic protection device 20. Thus, the tortuous pathway 30 is structured to permit the bodily fluid to follow the pathway 30 and pass beyond the device 20, while the tortuous pathway 30 is sufficient to prevent emboli from following the pathway 30 and passing beyond the device 20. For example, the device 20 is sized such that emboli will be unable to follow the tortuous path 30, while blood cells flowing through the blood vessel 10 are readily capable of following the tortuous pathway 30.

Also shown in FIG. 1 is a tether 34 which has been depicted as a wire guide. Many types of wire guides are known in the art, including coiled wire, solid wire, tubular wire, and combinations thereof, all of which can be employed as the tether 34, and further other materials such as plastic (preferably a biocompatible plastic) may also be readily employed in the tether 34. The tether 34 includes a distal cap 35 having a hook 36 projecting therefrom. The hub 22 of the embolic protection device 20 includes a proximal end 40 and a distal end 41, the proximal end 40 including a loop 38 which can be selectively connected to the hook 36 of the tether 34. It can also be seen that the hub 22 has been depicted as a hollow cylinder defining an inner lumen 26 which is sized to permit a wire guide or other device such as a catheter or the like, to be passed through the hub 22 of the embolic protection device 20. For example, a wire guide may be passed percutaneously into the body vessel 10 and located at a desired position, and then the hub 22 of the embolic protection device 20 may be placed over the wire guide and translated to the desired position. As shown in FIG. 4, either the proximal or distal ends 40, 41 of the elongated hub 22 may include a number of thin flaps 42 formed thereon in order to provide a seal at one end of the inner lumen 26 of the hub 22. In this manner, a wire guide may be used in conjunction with the embolic protection device 20, while at the same time emboli will be prevented from passing through inner lumen 26 of the device 20.

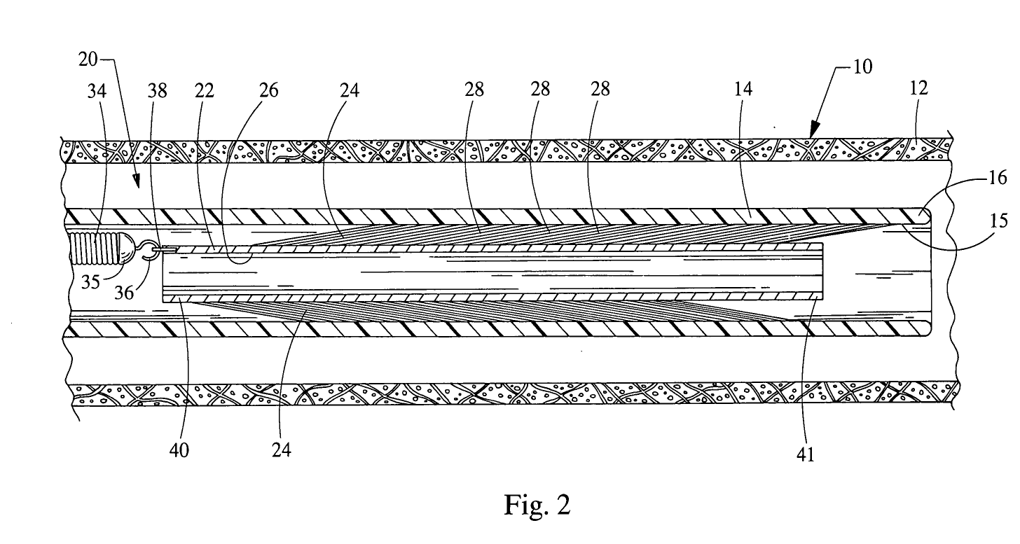

To place the embolic protection device 20 inside the body vessel 10, a delivery catheter 14 is preferably employed as shown in FIG. 2. The catheter 14 has a distal end 16 and defines a lumen 15 therethrough. The at least one projection 24, and particularly the plurality of bristles 28 have been shown in a loaded position in FIG. 2. It can thus be seen that the bristles 28 are operable between a loaded position and a deployed position, which are shown in FIGS. 2 and 1 respectively. The bristles 28 are formed with sufficient bending strength to return to the deployed position upon exiting the catheter 14. That is, the bristles 28 are biased to the deployed position but are forced into the loaded position by pulling the embolic protection device 20 into the catheter 14, preferably at the distal end 16 as shown. However, it will be recognized that the embolic protection device 20 may be loaded into the catheter 14 through a proximal end (not shown) in which case the bristles 28 would be angled proximally, rather than being angled distally as shown in FIG. 2. The latter loading condition would provide easier deployment of the embolic protection device which is achieved by longitudinally translating the catheter 14 relative to the device 20 and tether 34, or vice-versa. The hook 36 of the tether 34 may be detached and the embolic protection device 20 left in the body vessel 10 for a period of time, after which it could be retrieved by the tether 34 and then drawn into the catheter 14, wherein the bristles 28 would return to the loaded position shown in FIG. 2.

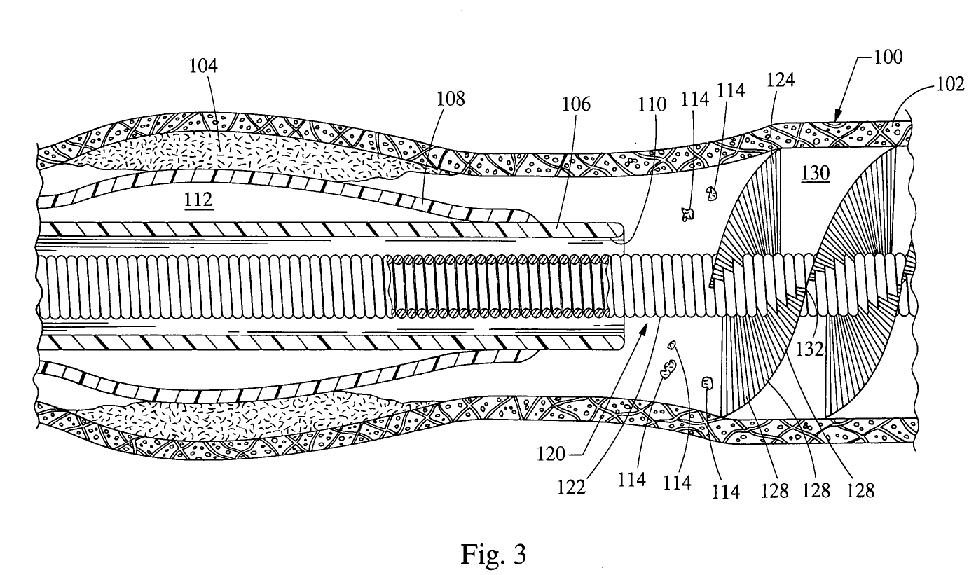

Another embodiment of an embolic protection device 120 has been depicted in FIG. 3. Generally, the embolic protection device 120 has been incorporated into a balloon catheter 106 to provide distal protection therefore. The construction of the balloon catheter 106 may take many forms as is well known to those skilled in the art. Generally, the balloon catheter 106 includes an outer expandable member 108 defining in a balloon cavity 112 between the member 108 and main catheter 106. As previously noted, such balloon catheters 106 are used to treat stenotic lesions 104 formed on the wall 102 of a body vessel 100. The balloon cavity 112 is filled with a gas (such as air) or liquid in order to fill the chamber 112 and inflate the expandable member 108 against the stenotic lesion 104. As is known, treatment of a stenotic lesion 104 in this manner can result in the release of emboli 114 which may be blood clots, plaque or the like.

In this embodiment, the embolic protection device 120 generally includes a hub 122 which also serves as the tether or actuating wire for the device 120. As in the prior embodiment, at least one projection 124 extends radially from the hub 122 and also extends longitudinally along the hub 122. Preferably, the at least one projection 24 comprises a plurality of bristles 128 attached to the hub 122 along a spiral path 132 to thereby form a tortuous pathway 130 between the hub 122 and the vessel wall 102. As such, the blood cells or other bodily fluid will be capable of following the spiral tortuous pathway 130, while emboli 114 will be trapped by the bristles 128. Once trapped, the relative translation of the elongated hub 122 and the catheter 106 will return the embolic protection device 120 to its loaded condition and the plurality of bristles 128 to their loaded position. As the bristles 128 enter the lumen 110 defined by the catheter 106, the bristles 128 will bend and move radially inwardly and will trap the emboli 114 within the bristles 128 and/or between the bristles 128 and the hub 122. The hub 122 is shown constructed of a coiled wire such as a wire guide, although may be constructed as a solid piece such as a tether, and may be constructed of either a metal or plastic material, as with the tether 34 of the prior embodiment.

It will be recognized by those skilled in the art that various other constructions of the embolic protection device 120, and in particular the structure of the tortuous pathway 130 will be readily envisioned by those skilled in the art while achieving the same embolic protection benefits. For example, the bristles 28, 128 could be constructed of differing lengths whereby the elongated hub 22, 122 may not be centered within the body vessel 100. The length of the bristles 28, 128 can vary circumferentially around the hub, 22, 122 or longitudinally along the hub 22, 122 (i.e. a tapered or conical shape). Likewise, the tortuous pathway 130 may not necessarily be spiral (or helical) but could include sharp bends or turns which still nonetheless define a tortuous pathway capable of preventing emboli from following the pathway 30, 130 and passing beyond the device 20, 120. Still further, two projections or two sets of bristles 28, 128 may be connected to the elongated hub 22, 122 to form such a pathway 30, 130, which could include a double helix configuration. It is understood that various designs may be constructed which utilize the principle of emboli tending to follow a laminar and centralized flow, while the bodily fluid such as blood is capable of following a more turbulent or tortuous pathway.

It will also be recognized by those skilled in the art that the entire embolic protection device 20 may be coated with an anti-thrombotic substance such as Heparin. That is, the hub 22 and projection 24 (namely the plurality of bristles 28) can be coated with the anti-thrombotic agent in order to reduce the potential for the device 20 from forming clots that might detach and become emboli.

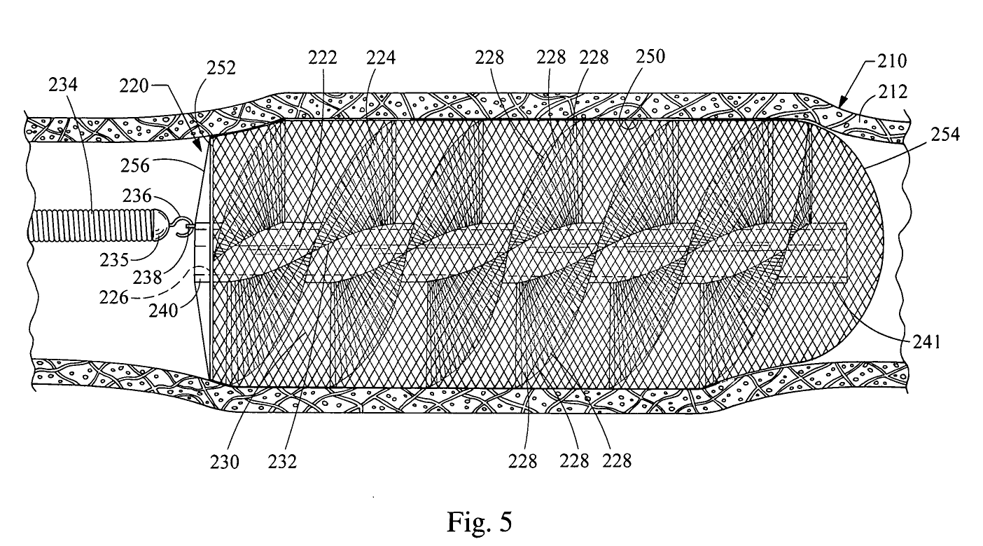

FIG. 5 depicts another embodiment of an embolic protection device 220 identical in most respects to the embodiment depicted in FIG. 1, including having a hub 222 with at least one projection 224 (namely a plurality of bristles 228) defining a tortuous pathway 230 and deployed/retrieved using a tether 234 having end cap 235 and hook 236 to engage loop 238 on proximal end 240 of hub 222. In this embodiment, the embolic protection device 220 includes a filter bag 250 extending around the periphery of the plurality of bristles 228. The filter bag 250 has been depicted as constructed of a mesh material (such as a medium density polyethylene), although numerous other filtering materials can be used as will be recognized by those skilled in the art. A proximal end 252 of the filter bag 250 is attached to the hub 222 by way of one or more struts 156. The distal end 154 of the filter bag 150 is downstream from distal end 240 of hub 222, and is closed in order to collect any emboli which may have become detached from the device 120, and in particular from the plurality of bristles 128. It will be recognized by those skilled in the art that upon retraction of the embolic protection device 120 into a catheter or sheath such as was depicted in FIG. 2 of the prior embodiment, the filter bag 150 will likewise collapse and be drawn into the inner lumen of the sheath.

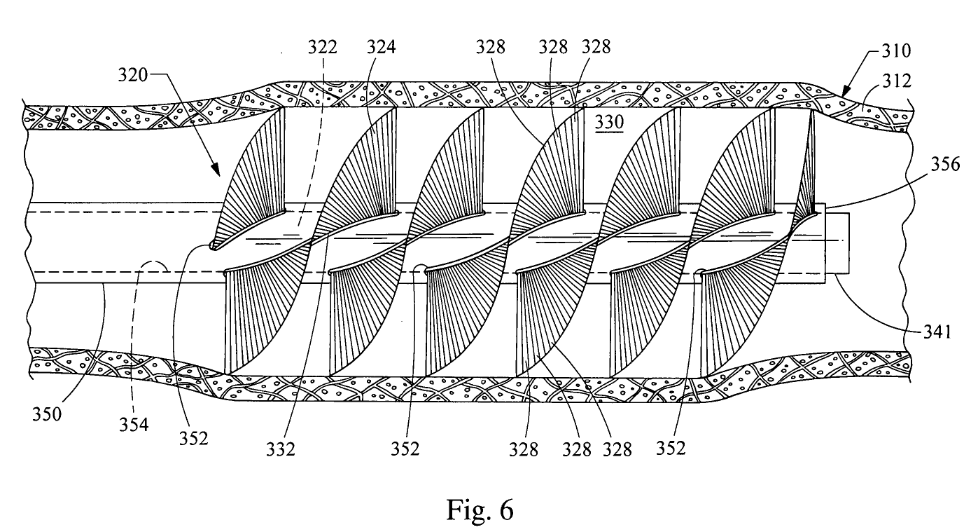

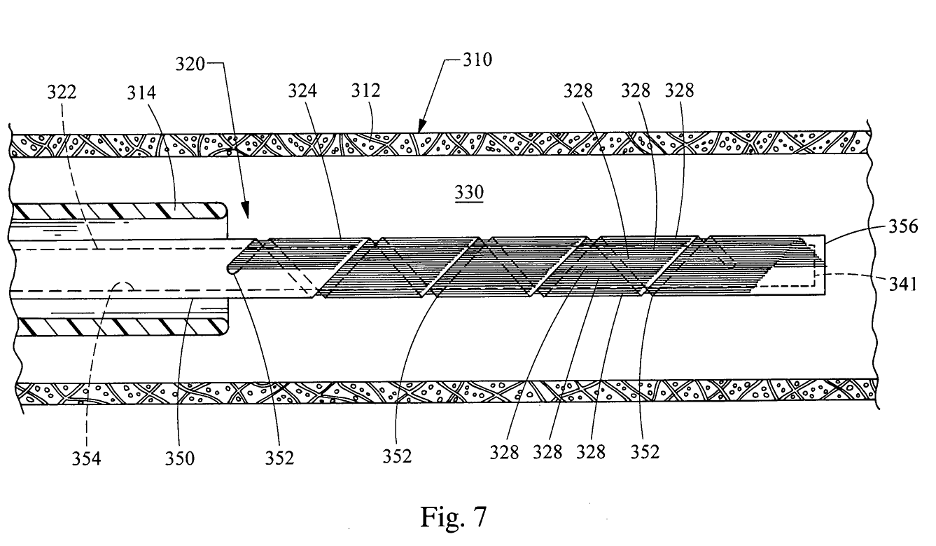

Yet another embodiment of an embolic protection device 320 has been depicted in FIGS. 6 and 7. This embodiment is also similar to the embodiment depicted in FIGS. 1 and 2, but includes an additional actuating member 350. Here, the projection 324, and in particular the plurality of bristles 328 need not be biased to either position, although the bristles 328 may be biased to the loaded position such as shown in FIG. 6.

In either case, the actuating member 350 defines an inner lumen 354 which is sized to receive the hub 322 of the embolic protection device 320. The hub 322 includes a plurality of bristles 328 adjacent a proximal end 341 thereof and defining a tortuous pathway 330 between the hub 322 (and/or the actuating member 350) and the wall 312 of vessel 310. The actuating member 350 includes one or more slots 352 formed therein which traverse a torturous path such as the spiral path formed by the plurality of bristles 328. As shown in FIG. 6, when the spiral slot 352 is longitudinally aligned with the spiral path 332 along which the plurality of bristles 328 are attached to the hub 322, the plurality of bristles 328 are forced into their deployed position as shown.

As shown in FIG. 7, the actuating member 350 may be translated longitudinally relative to the hub 322 and the plurality of bristles 328, whereby the spiral slot 352 is positioned relative to the spiral path 332 such that the plurality of bristles 328 are permitted to move to their loaded position as shown. The bristles 328 may be biased to their deployed position (FIG. 6) or may be biased to their loaded position (FIG. 7), or may be unbiased. When the plurality of bristles 328 are biased to their loaded position (FIG. 7), the spiral slot 352 is aligned with the spiral connection path 332 of the bristles 328 in the loaded condition, and in order to force the bristles to the deployed position, the actuating member 350 and hub 322 are relatively translated such that the edges of the spiral slot 352 press against the bristles 328 at the base thereof and along the spiral connection path 332. When the bristles 328 are biased to their loaded position (FIG. 6), the slot 352 is aligned with the path of the bristles 328 in the loaded condition, and non-aligned via the relative translation of member 350 and hub 322 to engage the base of bristles 328 and force them to their loaded position. When the bristles 328 are not biased in any particular manner, the edge of the spiral slot 352 will engage the bristles 328 in order to force the bristles into both their deployed and loaded conditions. Stated another way, the spiral slot 352 may become non-aligned with the spiral connection path 332 in order to force the bristles 328 into one of their loaded and deployed positions.

The foregoing description of various embodiments of the invention has been presented for purposes of illustration and description. It is not intended to be exhaustive or to limit the invention to the precise embodiments disclosed. Numerous modifications or variations are possible in light of the above teachings. The embodiments discussed were chosen and described to provide the best illustration of the principles of the invention and its practical application to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

1. An embolic protection device designed to be positioned inside a body vessel having bodily fluid, the device comprising:
an elongated hub;
at least one projection extending radially from the hub and extending longitudinally along the hub; and
the at least one projection defining a tortuous pathway between the hub and the vessel wall, the tortuous pathway structured to prevent emboli from following the pathway and passing beyond the device, the tortuous pathway structured to permit the bodily fluid to follow the pathway and pass beyond the device.

2. The device of claim 1, wherein the tortuous pathway takes a spiral shape.

3. The device of claim 1, wherein the at least one projection comprises a plurality of bristles attached to the hub.

4. The device of claim 3, wherein the plurality of bristles are attached to the hub along a spiral path.

5. The device of claim 1, wherein the at least one projection is operable between a loaded position and a deployed position.

6. The device of claim 5, wherein the at least one projection is biased towards the deployed condition.

7. The device of claim 1, wherein the bodily fluid is blood, and wherein the tortuous pathway structured to permit the blood cells to follow the pathway and pass beyond the device.

8. The device of claim 1, further comprising a balloon catheter, and wherein the hub and at least one projection provide distal protection for a balloon catheter.

9. The device of claim 8, wherein the hub and at least one projection fit with inner lumen of balloon catheter.

10. The device of claim 1, wherein the hub includes a lumen sized to receive a wire guide.

11. The device of claim 5, further comprising an actuating member translatable relative to the hub to control operation of the at least one projection between the loaded and deployed positions.

12. The device of claim 11, wherein the actuating member includes an inner lumen sized to receive the hub, and wherein the actuating member includes a slot structured to corresponds with a path formed at the connection between the hub and at least one projection.
